(19) United States
(12) Patent Application Publication
(10) Pub. No.: US 2023/0158066 A1
(43) Pub. Date: May 25, 2023

(54) ENCAPSULATED CARBONYL IRON SALT COMPOSITIONS AND PROCESS THEREOF

(71) Applicants: Pravin NALAWADE, Mumbai (IN); Vikrant SURYAVANSHI, Mumbai (IN)

(72) Inventors: Pravin NALAWADE, Mumbai (IN); Vikrant SURYAVANSHI, Mumbai (IN)

(21) Appl. No.: 17/770,404

(22) PCT Filed: Aug. 8, 2021
(86) PCT No.: PCT/IN2021/050759

§ 371 Date: Apr. 20, 2022

(30) Foreign Application Priority Data
Aug. 11, 2020 (IN) 202021034367

(51) Int. Cl. A61K 33/26; A61K 9/50; A23L 33/10; A23P 10/35

(52) U.S. Cl. CPC A61K 33/26; A61K 9/5015; A23L 33/10; A23P 10/35

(57) ABSTRACT

The composition of encapsulated carbinyl iron salt and a process for preparation thereof. The composition comprises high density particles of carbonyl iron salt in combination with lipids and/or emulsifiers. The invention also relates to composition may additionally contain other minerals or combination of minerals. The composition prevents side effects of the iron and masks metallic taste. Further, the composition provides an improved bioavailability and formulated into various oral delivery forms. This composition is useful in the prevention of iron deficiency anemia.

### FIELD OF THE INVENTION

The present invention relates to an encapsulated carbonyl iron salt composition and a process for preparation thereof. More particularly, the invention further relates to the encapsulated carbonyl iron salt composition comprises high density particles and encapsulating agent. The invention furthermore relates to the composition comprising high density particles and at least one or more lipid and/or emulsifier. The invention also relates to composition optionally contain other minerals or combination of minerals. The composition prevents side effects of the iron and masks metallic taste and released at the targeted site. Further, this improved bioavailable form of iron is formulated into various oral delivery forms. This composition useful in the prevention of iron deficiency such as anemia.

### BACKGROUND OF THE INVENTION

Malnutrition is a major issue is underdeveloped and developing countries. Due to insufficient dietary consumption mainly of nutrients such as vitamin A, ascorbic acid, folic acid, iron and iodine etc. More than billion children and women are suffering from malnutrition. Iron deficiency is the most common nutritional problem in the world, affecting two thirds of children in most of developing countries. Anaemia resulting from iron deficiency in young children has become very common since the level of bioavailable iron in a typical infant's diet is low while their rapid growth requires a much higher level of iron. The consequences of iron deficiency anaemia (IDA) are very serious as it is associated with impaired cognitive and psychomotor development, reduced growth and decreased resistance to infection. Such preparations are also used as nutritional supplements. Pregnant women are known to require significant dietary supplementation with iron, vitamins (e.g. folic acid), and non-ferrous minerals to minimize the risk of birth deformities, to improve the chances of a successful delivery and to improve birth weight of the foetus. In addition to their use in pregnant and lactating women, iron supplements containing vitamins, minerals, or both are well known and are used as sources of dietary iron to treat or prevent iron deficiency in mammals. These iron supplements generally include a single form of iron, for example, an iron (II) salt (i.e. a salt containing divalent or ferrous iron), an iron (III) salt (i.e. a salt containing trivalent or ferric iron), or iron (0) powder (e.g. carbonyl iron, typically made by heating gaseous iron pentacarbonyl, Fe(CO)₅. The iron in known supplements has been delivered in rapid release forms and in controlled release dosage forms.

US 20140234437A,1 disclosed invention which provides a composition comprising: (i) a non-encapsulated carbonyl iron salt, and (ii) a carrier comprising digestible edible oil that is a liquid at 20° C. This composition advantageously provides iron in a form that is easily administered to an infant or adult. When taken directly by mouth or added to food or infant formula, the composition has a desirably bland flavour and is easy to consume. Moreover, the high density of iron per unit volume of the composition results in a minimal volume of liquid to be administered. This makes the composition relatively innocuous and easy for infants to consume. Related aspects of the present invention are also described—e.g., uses, articles of manufacture and the like.

Iron sprinkles, a powder containing micro-encapsulated ferrous fumarate is also known in the prior art. For example, U.S. Pat. No. 6,830,761 [Zlotkin] teaches a composition useful in the prevention of iron deficiency anaemia. The composition comprises micro-encapsulated iron granules in combination with a lipid-based excipient. The composition may additionally contain other micronutrients including ascorbic acid, zinc, vitamin A and iodine. Zlotkin teaches that the composition is particularly useful for the prevention of iron deficiency anemia in infants between the ages of 6 and 24 months of age since it can readily be admixed with the semi-solid foods this age group consumes. The importance of using micro-encapsulated iron (i.e., versus non-encapsulated iron) is illustrated in Examples 1 and 2 of Zlotkin. Specifically, it is reported in Example 2 of Zlotkin that there is no significant difference in the hemoglobin response between rats fed similar amounts of iron as the reference compound (i.e., non-encapsulated ferrous sulfate) versus rats fed micro-encapsulated ferrous fumarate. Thus, the use of micro-encapsulated iron is central to the teaching of Zlotkin.

U.S. Pat. No. 3,992,556 [Kovacs et al. (Kovacs)] teaches a particulate food supplement composition comprising a nutrient such as assimilable iron compounds, vitamins, minerals or mixtures thereof which nutrient is uniformly dispersed in a carrier consisting essentially of particles or beads of an edible metabolizable fat. Notably, the edible fat is solid at room temperature, with a preferred softening or melting point between about 100° and 250° F., whereby the assimilable iron compounds, vitamins, minerals or mixtures thereof can be conveniently added or applied to a variety of foods, such as breakfast food cereals, crackers, cookies, potato chips and similar snack foods, flour and pasta during their productions. The particulate food supplement taught by Kovacs is not suitable for use a liquid (room temperature) nutrient supplement, let alone a liquid (room temperature) iron nutrient supplement.

Prior art controlled release dosage forms of iron-containing nutrients.

Nutritional supplements have generally used an iron (II) salt encapsulated in or mixed with a release rate modifying matrix, one of certain iron (III) salts which exhibit poor solubility, carbonyl iron or one of the other metallic forms of iron (which also exhibit poor solubility), a certain crystalline form of iron oxide, or an iron salt or carbonyl iron complexed with a protein, an amino acid, an organic acid, a natural polymer, an anionic complexing agent, or a synthetic polymer. Administration of known controlled release dosage forms generally results in temporary reductions of blood iron levels between consecutive doses. These temporary reductions can be due to the tapered delivery rate of iron from a first dose coupled with a delayed, or slow initial, delivery of iron from a second dose. At least certain supplements designed to provide sustained delivery of iron have been associated with unpleasant tastes and odors, nausea, stomach irritation, and gas formation (e.g. manifested as eructation).

U.S. Pat. No. 6,468,568B1 disclosed, a mineral or vitamin fortification ingredient which does not deleteriously affect palatability and appearance of foods is obtained by encapsulation of the mineral or vitamin in a grindable, glassy matrix composition. The glassy matrix composition includes an oligosaccharide, such as β-2-1 fructofuranose materials, preferably fructooligosaccharides (FOS) and inulin, which not only forms a glassy matrix, but also beneficially increases the fibre content of the food. At least one edible oil is included in the encapsulating composition to prevent substantial adverse interaction between the mineral or vitamin encapsulant and the glass-forming oligosaccharide matrix material, and to achieve controlled release of the encapsulant from the glassy matrix.

There is a significant, continuing need for an iron supplement which can be administered to a human patient in order to safely and efficaciously deliver a nutritionally relevant amount of iron to the patient. In view of safety (e.g. accidental poisoning) and patient compliance issues (e.g. failure to consistently ingest iron supplements, owing to unpleasant side effects, unpleasant taste or odor, inconvenient tablet size, or some combination of these) which exist with regard to prior art iron supplements, a particular need remains for an iron supplement having a reduced risk of accidental poisoning, reduced side effects, and greater patient acceptance, which can lead to improved patient compliance with a dosing regimen. The present invention satisfies these needs.

### OBJECTIVES OF THE INVENTION

The main objective of the present invention is to provide an encapsulated carbonyl iron salt composition.

The further objective of the present invention is to provide encapsulated carbonyl iron salt composition comprising a high-density particles and encapsulating agent.

Yet another object of the present invention is to provide a process for encapsulating carbonyl iron salt using lipids and/or emulsifiers.

The further objective of the present invention is to provide encapsulated carbonyl iron salt composition in the form of granules.

Still another object of the present invention is to provide an encapsulated carbonyl iron salt composition to be used in sprinkles, micronutrient premixes, tablets, capsules and food fortification like salt, rice and wheat flour etc.

Another objective of the present invention is to provide slow release encapsulated carbonyl iron salt composition.

Another object of the present invention is to provide encapsulated carbonyl iron salt composition which is non-reactive, minimize metallic taste and odor.

Further objective of the present invention is to provide encapsulated carbonyl iron salt composition for oral delivery with no side effects, gastric irritation, nausea and vomiting.

Furthermore, objective of the present invention is to provide encapsulated carbonyl iron salt composition useful to the prevention and treatment of iron deficiency anemia.

### BRIEF DESCRIPTION OF ACCOMPANYING FIGURES

FIG. 1: Shows Iron release in profile (%) from Ferrous fumarate composition (%) as per Example 01.

FIG. 2: Shows Iron release in profile (%) from Ferrous fumarate composition (%) as per Example 02.

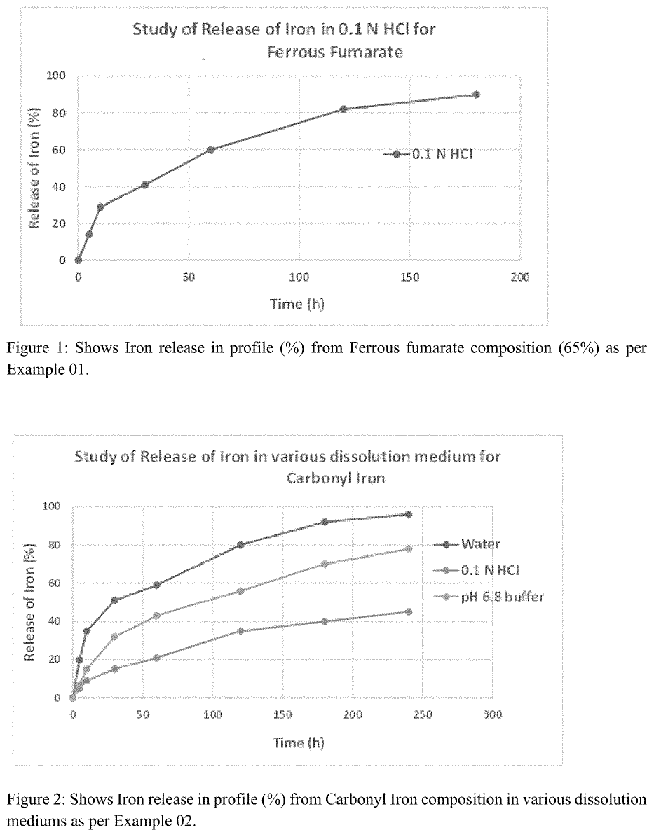

### SUMMARY OF THE INVENTION

The present invention relates to an encapsulated carbonyl iron salt composition and a process for preparation thereof.

The process for preparation of encapsulated carbinyl iron salt composition comprising the steps of:
(i) Weigh all the ingredients.
(ii) Transfer carbonyl iron salt powder with particles density 0.1 to 10 g/mL in fluid bed processor after passing through 100 #mesh.
(iii) Melt lipid and/or emulsifier to form flowable liquid by heating.
(iv) Spray molten flowable mixture on carbonyl iron to form uniform granules.
(v) Unloaded the granules and pass-through sieve and pack.

The composition of high-density particles encapsulated with lipids and/or emulsifiers.

The encapsulated composition of iron of carbonyl iron salt used in sprinkles, micronutrient premixes, tablets, capsules and food fortification like salt, rice and wheat flour etc. but not restricted to all these application in food, pharmaceutical and nutrition.

### DETAILED DESCRIPTION OF THE INVENTION

The invention described herein relates to the encapsulated carbonyl iron salt composition and a process for preparation thereof.

Within the context of this invention the terminology the “encapsulated carbonyl iron salt composition” is commonly used in the specification to refer composition having selectively high-density particles of carbonyl iron salt of bulk density 0.1 to 10 g/mL and encapsulating agent.

According to embodiment of the present invention the encapsulated carbonyl iron salt composition prepared by encapsulation using lipids and/or emulsifiers.

According to another embodiment relates to a composition of encapsulated carbonyl iron salt useful to the prevention and treatment of iron deficiency anemia.

The carbonyl iron salts in the present invention are obtained by conventional or non-conventional synthesis methods known to a person skilled in art.

Encapsulation techniques have been used to coat reactive minerals. The protective mechanism is to form a membrane, the wall system, around particles of the encapsulated material, the core. Encapsulation not only protects against losses and chemical changes, but also enables production in the form of powdered products with new properties like taste masking, odor masking and avoid side effects.

The prior arts are speaking on nutritional supplements containing a rapid release dosage form of iron have generally contained a rapidly dissolving iron salt and normal iron salt varied in oxidizing capacity since certain iron salts are significantly more soluble in water and in gastrointestinal fluids than other salts and metallic forms of iron. These formulations can cause unpleasant, harmful, or even fatal side effects. By way of example, such side effects include stomach irritation, constipation, and iron poisoning. Toxic side effects of iron can be attributed to the high solubility and the high dissolution rate of certain iron salts (e.g. ferrous sulphate) in the gastrointestinal tract. The incidence of accidental iron poisoning (e.g. by young children who ingest prenatal vitamin supplements). So, there is strong need to provide the composition of carbonyl iron salt of high-density particles which having the properly encapsulated to avoid the above side effects. Hence this instant invention identified the need and invented the encapsulation technique for high-density particles with suitable excipients with the targeted release profile in stomach at different pH.

In an embodiment, the high-density particles of carbonyl iron salt are selected from the group consisting of carbonyl iron, such as carbonyl iron is a highly pure iron, prepared by chemical decomposition of purified iron pentacarbonyl. It usually has the appearance of grey powder, composed of spherical microparticles. In pharmaceutics, carbonyl iron powder is used to treat iron deficiency and as an iron dietary supplement.

In an another embodiment, the high-density particles of carbonyl iron salt are selected from the group consisting of ferrous fumarate, ferrous succinate, ferrous gluconate, ferric pyrophosphate, ferric saccharate, ferric orthophosphate, ferrous ascorbate, ferrous sulfate alone or mixtures of one or more of these with minerals zinc oxides, copper sulphates, zinc sulphate but not limited.

According to further embodiment the high-density particles of carbonyl iron salt present is in the range of 1%-95% w/w of total the composition.

According to further embodiment the high-density particles of carbonyl iron salt is present in the range of 5%-99% w/w of total the composition.

In preferred embodiment the encapsulating agents used in the composition of the present invention is selected from lipids such as hydrogenated fats, phospholipids, natural waxes, gums, polyethylene glycols, oligosaccharides alone or mixtures thereof.

In preferred embodiment the encapsulating agents used in the composition of the present invention is selected from solid emulsifiers such as glyceryl mono-stearate, glyceryl di-stearates, lecithin, steric acid, sucrose ester gums, sorbitan monoesters, sorbitan mono oleates alone or mixtures thereof.

According to another embodiment, the encapsulating agent is present in the range of 1-95% w/w of total the composition.

According to preferred embodiment, the encapsulating agent is present in the range of 5-99% w/w of total the composition.

According to another embodiment, the carbonyl iron salt has apparent density in the range of 0.1-10 g/mL

According to another preferred embodiment carbonyl iron salt has apparent density in the range of preferably 1-6 g/mL

According to further embodiment, the carbonyl iron salt comprises from the group consisting of iron salt which comprises the iron salt compound having carbonyl group and has apparent density in the range of 0.1-10 g/mL, but, preferably 1-6 g/mL.

According to further embodiment of the present invention encapsulated particles comprising high density particles of carbonyl iron salt and lipid and/or emulsifier in preferred percentage which may be formulated in different forms for oral delivery. The dosage form maybe selected from sprinkles, micronutrient premixes, tablets, capsules, powders, granules, pellets, beadlets and food fortification like salt, rice and wheat flour etc. but not restricted to all these application in food, pharmaceutical and nutrition.

From the details given above it can be observed that the carbonyl iron salt composition of the present invention is not a mere admixture resulting in a composition which having the aggregation of the properties of the components used but a composition formed with the synergistic activities of the components used.

In still another embodiment the dose of encapsulated particles is in the range of 5 mg to 1000 mg.

More preferably the dose of encapsulated particles is in the range of 50 mg to 250 mg.

In another embodiment of the present invention, the encapsulated iron composition is prepared by a method comprising:
(vi) Weigh all the ingredients.
(vii) Transfer carbonyl iron salt powder with particles density 0.1 to 10 g/mL in fluid bed processor after passing through 100 #mesh.
(viii) Melt lipid and/or emulsifier to form flowable liquid by heating.
(ix) Spray molten flowable mixture on carbonyl iron to form uniform granules.
(x) Unloaded the granules and pass-through sieve and pack.

In a preferred embodiment, the spraying process of present invention is carried out by using bottom spray, top spray fluid bed processor or by tangential spray, top spray Flex Stream process or pan coating.

The following examples are for the purpose of illustration of the invention only and are not intended in any way to limit the scope of the present invention.

### Examples of the Invention

The present invention is now illustrated by means of non-limiting examples and the problems associated in prior art on the coating of high-density particles when the materials like iron salt and/or carbonyl iron salt which exhibit the unpleasant test. The present invention mainly addresses this issued with high density particle coating to material like carbonyl iron salt.

The coatings on high density particles are a complex combination of type of material with that must be mixed, applied to a prepared substrate, and dried and cured correctly to perform to their maximum capability. They must be able to be applied in diverse environmental conditions and then be expected to protect the substrate from the damaging effects of effect of external factors in various combinations of media in stomach and cycles and still retain their integrity and often their aesthetic qualities. It is not generally straightforward to establish the reason for the failure of a coating due to the many potential factors that may be involved. These could include formulation, types of material, density of particles, application, drying and curing times and conditions, and environmental exposure, with more than one contributing factor often being involved. One of the difficulties associated with the prior art mainly approaches to adequate iron supplementation is the poor adherence of people to taking an iron supplement. This is partly the result of the unpleasant nature a currently available to iron products so masking or coating may be the essential part. However, the coating on high density particles of carbonyl iron salt is also a very cumbersome and required to encapsulate properly with proficient encapsulation technique as the Iron has an unpleasant taste. So, this instant invention came up with solution mainly on high density particles encapsulation as per the forgoing example but not limited to this.

Particle density or Bulk density is the density of the solids (determined by the composition), which determines the particle density, together with the amount of occluded air. It's the ratio of mass to volume (including the inter-particulate void volume) of an untapped powder sample. It depends on both the density of powder particle and arrangement of the particles. The bulk density influenced by preparation method, treatment and storage of the samples.

The method used as per the reference of USP general chapter <616> method 1.
1. Pass a quantity of material sufficient to complete the test through sieve greater than equal to 1 mm, if necessary to break up agglomerates that may formed.
2. Fill the test sample approx. 150 g with 0.1% accuracy (W) in dry gradual 250 mL cylinder (readable to 2 mL) carefully.
3. Read the unsettled apparent volume (V0) to the nearest graduated unit.
4. Calculate the particle density by following formula;

Particle density=W/V0

Particle density of various carbonyl iron salt.

| Sr. No. | Active Name | Particle density (g/mL) |
|---|---|---|
| 1 | Carbonyl Iron | 4.42 |
| 2 | Ferrous fumarate | 2.14 |
| 3 | Ferrous sulfate | 1.26 |
| 4 | Ferrous Ascorbate | 0.75 |

Carbonyl iron is a highly pure iron, prepared by chemical decomposition of purified iron pentacarbonyl. It usually has the appearance of grey powder, composed of spherical microparticles. In pharmaceutics, carbonyl iron powder is used to treat iron deficiency and as an iron dietary supplement.

### Example 1

### Formulation: Granules

| | | Quantity of | |
|---|---|---|---|
| Sr. No. | Ingredients | Ingredients (g) | % w/w |
| 1. | Ferrous Fumarate (particle | 650.00 | 65 |
| | density 2.14 g/mL) | | |
| 2. | glyceryl mono di glyceride | 350.00 | 35 |

### (A) Process:

The process for preparation of composition has defined in the stepwise manner as follows:
(i) Weigh all the ingredients as provided in table.
(ii) Transfer Ferrous fumarate particles having density 2.14 g/mL in fluid bed processor after passing through 80 #mesh.
(iii) Melt glyceryl mono and di stearate in provided quantity by heating upto 70° C.
(iv) Spray molten glyceryl mono di glyceride on ferrous fumarate salt to form uniform coated granules.
(v) Unloaded the granules and pass through 50 #mesh and pack.

### (B) Dissolution Profile:

Dissolution study of granules obtained from example 1 (encapsulated ferrous fumarate salt composition) was performed using a USP II paddle apparatus.
a. Dissolution medium: 0.1N HCl
b. Dissolution medium volume: 900 ml at each stage
c. Type: USP Type II
d. RPM: 50
e. Time: 3 h
f. Sampling Interval: As per the table.
Analysis of Iron (Fe⁺²) was done using titration method.

TABLE 01
Dissolution data of encapsulated Ferrous Fumarate
salt composition as per Example 01

| | % Release of Iron from |
|---|---|
| Time (Min) | Ferrous Fumarate granules |
| 0 | 0 |
| 5 | 14 |
| 10 | 29 |
| 30 | 41 |
| 60 | 60 |
| 120 | 82 |
| 180 | 90 |

### Example 02

### Formulation: Granules

| | | Quantity of | |
|---|---|---|---|
| S. NO | INGREDIENTS | Ingredients (g) | % w/w |
| 1. | Carbonyl Iron (particle density | 650.00 | 65 |
| | 4.42 g/mL) | | |
| 2. | Hydrogenated palm oil | 300.00 | 30 |
| 3. | Sunflower Lecithin | 50.0 | 5 |

(A) Process:

The process for preparation of composition has defined in the stepwise manner as follows:
(i) Weigh all the ingredients as provided in above table.
(ii) Transfer carbonyl iron salt in fluid bed processor after passing through 80 #mesh.
(iii) Melt hydrogenated palm oil and sunflower lecithin by heating upto 70° C.
(iv) Spray molten hydrogenated palm oil and sunflower lecithin on carbonyl iron salt to form uniform granules.
(v) Unloaded the granules and pass through 30 #mesh and pack.

(B) Dissolution Profile:

Dissolution study of encapsulated carbonyl iron salt composition was performed using a USP II paddle apparatus.
a. Dissolution medium: water, 0.1N HCl and pH 6.8 buffer separately
b. Dissolution medium volume: 900 mL at each stage
c. Type: USP Type II
d. RPM: 50
e. Time: 3 h
f. Sampling Interval: As per above table.

Analysis of Iron (Fe⁺²) was done using titration method.

TABLE NO 02
Dissolution data of encapsulated carbonyl
iron salt composition as per Example 02

| | | % Release of | % Release of |
|---|---|---|---|
| | % Release of | Carbonyl Iron | Carbonyl Iron |
| Time | Carbonyl Iron | granules in | granules in pH |
| (min) | granules in water | 0.1N HCl | 6.8 buffer |
| 0 | 0 | 0 | 0 |
| 5 | 20 | 5 | 7 |
| 10 | 35 | 9 | 15 |
| 30 | 51 | 15 | 32 |
| 60 | 59 | 21 | 43 |
| 120 | 80 | 35 | 56 |
| 180 | 92 | 40 | 70 |
| 240 | 96 | 45 | 78 |

### Example 03

### Formulation: Granules

| | | Quantity of | |
|---|---|---|---|
| | | Ingredients | |
| Sr. NO | INGREDIENTS | (grams) | % w/w |
| 1. | Ferrous Fumarate (particle | 650.00 | 65 |
| | density 2.14 g/mL) | | |
| 2. | Glyceryl mono and di stearate | 350.00 | 35 |

The process for preparation of composition has defined in the stepwise manner as follows:
(i) Weigh all the ingredients.
(ii) Transfer Ferrous Fumarate salt powder (particle density 2.14 g/mL) in fluid bed processor after passing through 80 #mesh.
(iii) Melt Glyceryl mono and di stearate by heating upto 70° C.
(iv) Spray molten glyceryl mono di glyceride on Ferrous Fumarate to form uniform granules.
(v) Unloaded the granules and pass through 30 #mesh and pack.

### Example 04

### Formulation: Granules

| | | Quantity of | |
|---|---|---|---|
| S. NO | INGREDIENTS | Ingredients (g) | % w/w |
| 1. | Carbonyl Iron salt (particle | 850.00 | 85 |
| | density 4.42 g/mL) | | |
| 2. | Hydrogenated Palm Oil | 150.00 | 15 |

(i) The process for preparation of composition has defined in the stepwise manner as follows: Weigh all the ingredients.
(ii) Transfer carbonyl iron salt in fluid bed processor after passing through 80 #mesh.
(iii) Melt hydrogenated palm oil by heating upto 70° C.
(iv) Spray molten hydrogenated palm oil on carbonyl iron salt to form uniform granules.
(v) Unloaded the granules and pass through 30 #mesh and pack.

### Example 05

### Formulation: Granules

| | | Quantity of | |
|---|---|---|---|
| S. NO | INGREDIENTS | Ingredients (g) | % w/w |
| 1. | Ferrous sulphate (Particle | 650.00 | 65 |
| | density - 1.26 g/mL) | | |
| 2. | Hydrogenated Soya oil | 350.00 | 35 |

The process for preparation of composition has defined in the stepwise manner as follows:
(i) Weigh all the ingredients.
(ii) Transfer Ferrous sulphate in fluid bed processor after passing through 60 #mesh.
(iii) Melt hydrogenated soya oil by heating upto 70° C.
(iv) Spray molten hydrogenated soya oil on Ferrous sulphate to form uniform granules.
(v) Unloaded the granules and pass through 30 #mesh and pack.

### Example 06

### Formulation: Granules

| | | Quantity of | |
|---|---|---|---|
| | | Ingredients | |
| S. NO | INGREDIENTS | (grams) | % w/w |
| 1. | Ferrous Sulphate (Particle | 650.00 | 65 |
| | density - 1.26 g/mL) | | |
| 2. | Stearic Acid | 350.00 | 35 |

The process for preparation of composition has defined in the stepwise manner as follows:
(i) Weigh all the ingredients.
(ii) Transfer Ferrous Sulphate in fluid bed processor after passing through 60 #mesh.
(iii) Melt Stearic acid by heating upto 80-90° C.
(iv) Spray molten Stearic acid on Ferrous Sulphate to form uniform granules.
(v) Unloaded the granules and pass through 30 #mesh and pack.

### Example 07

### Formulation: Granules

| | | Quantity of | |
|---|---|---|---|
| | | Ingredients | |
| S. NO | INGREDIENTS | (grams) | % w/w |
| 1. | Carbonyl Iron (min 97% | 200.00 | 20 |
| | Iron content) | | |
| 2. | Hydrogenated Palm Oil | 450.00 | 40 |
| 3. | Mono & di glyceride | 450.00 | 40 |
| | mixture | | |

The process for preparation of composition has defined in the stepwise manner as follows:
(i) Weigh all the ingredients.
(ii) Transfer Carbonyl Iron in coating pan after passing through 80 #mesh.
(iii) Melt hydrogenated palm oil and Mono & di glycerides by heating upto 90-100° C.
(iv) Spray molten hydrogenated palm oil and Mono & di glycerides on carbonyl iron to form uniform granules.
(v) Unloaded the granules and pass through 30 #mesh and pack.

### Example 08

### Formulation: Granules

| | | Quantity of | |
|---|---|---|---|
| | | Ingredients | |
| S. NO | INGREDIENTS | (grams) | % w/w |
| 1. | Ferrous Ascorbate (Particle | 650.00 | 65 |
| | density 0.75 g/mL) | | |
| 2. | Hydrogenated Soya oil | 350.00 | 35 |

The process for preparation of composition has defined in the stepwise manner as follows:
(i) Weigh all the ingredients.
(ii) Transfer Ferrous Ascorbate in fluid bed processor after passing through 40 #mesh.
(iii) Melt hydrogenated soya oil by heating upto 100° C.
(iv) Spray molten hydrogenated soya oil on Ferrous Ascorbate to form uniform granules.
(v) Unloaded the granules and pass through 20 #mesh and pack.

We claim:

1. An encapsulated carbonyl iron salt composition comprises:
i) A high-density particle in the range of 0.1-10 g/mL and weight to the composition in the range of 1-95% w/w; and
ii) Encapsulating agent in the range of 5-99% w/w.

2. The encapsulated carbonyl iron salt composition as claimed in claim 1, wherein the high-density particles are of carbonyl irone salt selected from carbonyl iron, ferrous fumarate, ferrous succinate, ferrous gluconate, ferric pyrophosphate, ferric saccharate, ferric orthophosphate, ferrous ascorbate, ferrous sulfate and mixtures thereof.

3. The encapsulated carbonyl iron salt composition as claimed in claim 1, wherein encapsulating agent is selected from lipids and emulsifiers or mixtures thereof.

4. The encapsulated carbonyl iron salt composition as claimed in claim 3, lipid is selected from hydrogenated fats, phospholipids, natural waxes, gums, polyethylene glycols, oligosaccharides alone or mixtures thereof.

5. The encapsulated carbonyl iron salt composition as claimed in claim 3, wherein the emulsifier is selected from glyceryl mono-stearate, glyceryl di-stearates, lecithin, sucrose ester gums, sorbitan monoesters, sorbitan mono oleates alone or mixtures thereof.

6. The encapsulated carbonyl iron salt composition as claimed in claim 1, wherein the composition is used in sprinkles, micronutrient premixes, tablets, capsules, powders, pellets, beadlets and food fortification like salt, rice and wheat flour.

7. The encapsulated carbonyl iron salt composition as claimed in claim 1, wherein the composition is in the form of granules.

8. The encapsulated carbonyl iron salt composition as claimed in claim 1, wherein the release rate of encapsulated carbonyl iron salt composition in water, 0.1 N HCl and in pH 6.8 are:

| | | % Release of | % Release of |
|---|---|---|---|
| | % Release of | Carbonyl Iron | Carbonyl Iron |
| Time | Carbonyl Iron | granules in | granules in pH |
| (min) | granules in water | 0.1N HCl | 6.8 buffer |
| 0 | 0 | 0 | 0 |
| 5 | 20 | 5 | 7 |
| 10 | 35 | 9 | 15 |
| 30 | 51 | 15 | 32 |
| 60 | 59 | 21 | 43 |
| 120 | 80 | 35 | 56 |
| 180 | 92 | 40 | 70 |
| 240 | 96 | 45 | 78 |
